(19) United States
(12) Patent Application Publication
(10) Pub. No.: US 2022/0039677 A1
(43) Pub. Date: Feb. 10, 2022

(54) METHODS AND APPARATUSES FOR DETERMINING FATIGUE INDEX

(71) Applicant: TAIPEI MEDICAL UNIVERSITY, Taipei City (TW)

(72) Inventors: Tsai-Wei HUANG, Taichung City (TW); Chi-Huang SHIH, Taichung City (TW); Pai-Chien CHOU, New Taipei City (TW)

(21) Appl. No.: 17/377,149

(22) Filed: Jul. 15, 2021

Related U.S. Application Data
(60) Provisional application No. 63/060,839, filed on Aug. 4, 2020.

(51) Int. Cl. A61B 5/024; A61B 5/00

(52) U.S. Cl. CPC A61B 5/02405; A61B 2560/0475; A61B 5/72

(57) ABSTRACT

The present disclosure provides methods and apparatuses for determining a fatigue index. A method of determining a fatigue index may include receiving physiological signals, generating a plurality of parameters of heart rate variability based on the physiological signals, and determining the fatigue index based on the plurality of parameters of heart rate variability.

### CROSS-REFERENCE TO RELATED APPLICATIONS

This application claims priority to U.S. Provisional Application No. 63/060,839 filed on Aug. 4, 2020, entitled “METHOD AND SYSTEM FOR MEASURING CANCER-RELATED FATIGUE,” the contents of which is incorporated herein in its entirety.

### BACKGROUND OF THE INVENTION

Cancer-related fatigue (CRF) is a serious side effect of cancer and its treatment that can disrupt the quality of life of the patients. Clinically, the standard method for assessing CRF relies on the subjective experience retrieved from patient self-reports such as Brief Fatigue Inventory (BFI). However, most patients do not self-report their fatigue level.

Most subjects may not clearly understand that they suffer from fatigue. Oftentimes, subjects may be more fatigued than they realize. For normal subjects, fatigue may be caused by various conditions such as arrhythmia, hypothyroidism, kidney diseases, liver diseases, or depression. Recognizing fatigue can be helpful in identifying subsequent conditions.

### BRIEF DESCRIPTION OF THE DRAWINGS

Aspects of the present disclosure are best understood from the following detailed description when read with the accompanying figures. It is noted that in accordance with the standard practice in the industry, various features are not drawn to scale. In fact, the dimensions of the various features may be arbitrarily increased or reduced for clarity of discussion.

FIG. 1 is a flowchart of a process of determining a fatigue index (FI) in accordance with some embodiments of the present disclosure.

FIG. 2 is a flowchart of a process of generating parameters of heart rate variability (HRV) in accordance with some embodiments of the present disclosure.

FIG. 3 shows curves of the parameters of HRV in accordance with some embodiments of the present disclosure.

FIG. 4 is a flowchart of a process of determining a FI in accordance with some embodiments of the present disclosure.

FIG. 5 depicts the relationship between the BFI and the FI in accordance with some embodiments of the present disclosure.

FIG. 6 is a flowchart of a process of updating factors in accordance with some embodiments of the present disclosure.

FIG. 7 is a flowchart of a process of determining factors in accordance with some embodiments of the present disclosure.

FIG. 8 depicts the relationship between the BFI and the LF/HF disorder ratios in the sleep phase in accordance with some embodiments of the present disclosure.

FIG. 9 depicts the relationship between the BFI and the average values of LF/HF ratios in the sleep phase in accordance with some embodiments of the present disclosure.

FIG. 10 depicts the relationship between the BFI and the LF/HF disorder ratios in the active phase in accordance with some embodiments of the present disclosure.

FIG. 11 is a diagram of a system in accordance with some embodiments of the present disclosure.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on, or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operations or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wireless connected, coupled, or linked with each other.

As used herein, the singular terms “a,” “an,” and “the” may include plural referents unless the context clearly indicates otherwise. For example, reference to a device may include multiple devices unless the context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

Fatigue may be an indicator of the need for energy reimbursement in normal individuals. Cancer-related fatigue (CRY), defined as a multidimensional phenomenon that develops over time, can be characterized by diminished energy and mental capacity and disturbed psychological condition among patients with cancer. CRY prevalence may be estimated to vary from 60% to 90%, depending on the diagnostic criteria used. CRF may often coexist with symptoms of depression, pain, anorexia, insomnia, anxiety, nausea, and dyspnea, all of which can contribute to the expression of fatigue. CRF may impose limitations on the normal daily activities of patients with cancer and profoundly affects all aspects of their quality of life, including compliance with standard treatments such as chemotherapy or radiotherapy. Thus, CRF management may be an essential part of a treatment plan for patients with cancer.

To evaluate the severity of CRF, patients in most studies may be subjectively evaluated through questions such as “How would you rate your fatigue on a scale of 0 to 10 over the past 7 days?” (0=no fatigue, 10=worst fatigue you can imagine). Additional measurements can be conducted using other standardized assessment tools such as the Brief Fatigue Inventory (BFI). Brief Fatigue Inventory (BFI) may be based on the subjective experience retrieved from patient self-reports such. According to guidelines, regular screening of CRF in clinical practice is suggested. However, in reality, CRF may often remain underreported and go untreated for a variety of reasons. First, most methods of measuring CRF can be subjective. Second, the patients may gradually become accustomed to their impaired physical condition and consider the discomforts normal. Third, while several pharmaceutical remedies have been used in clinical practice to relieve CRF, no standard treatments thus far have been promoted in clinical guidelines or have proven effective.

Heart rate variability (HRV) may be measured using a method similar to electrocardiography (ECG). HRV may give the indication of variability between two adjacent heartbeats. HRV may be continuously modulated through complex interactions by the autonomic nervous system, involving the sympathetic nervous system (SNS), parasympathetic nervous system (PNS), and vagus nerve. Activities of the SNS increase heart rate (HR), whereas those of the PNS reduce it. HRV may include time domain and frequency domain parameters. Frequency-domain HRV parameters may detect fatigue and its corresponding effects such as drowsiness and sleep.

Current frequency-domain measurements assign bands of frequency into a high frequency (HF) band ranging between 0.14 and 0.4 Hz and low frequency (LF) band ranging between 0.05 and 0.15 Hz. The ratio of LF power to HF power (LF/HF) represent the relative activity between the SNS and PNS under controlled conditions. A decrease in HF power and an increase in LF power can be characterized as low parasympathetic activity.

Stress may affect the SNS, PNS, and vagus nerve activities. Thus, HRV may indicate the psychological health status of patients. HRV may measure fatigue in different scenarios and diverse disease groups. HRV may identify waking and fatigue states.

HF power may increase during a fatigue state, whereas LF power may increase during waking state. In non-rapid-eye-movement (non-REM) states, the LF/HF ratio may gradually decrease as sleep deepens. The LH/HF ratio may reflect sleep activity. LF/HF may differentiate between non-REM and REM states. Sleep quality at night and rest frequency during daytime may potentially correspond to fatigue status. A higher fatigue level may cause higher rest frequency during the daytime, affecting sleep quality at night.

Sleep activity includes REM and non-REM states. The non-REM state may consist of stages 1, 2, 3, and 4, in larger numbers may indicate deeper sleep. Dreams typically occur during the REM state, whereby the brain becomes more active than in non-REM state. A sleep cycle may initiate with the non-REM state, starting from non-REM stages 1 to 4 and back to non-REM stage 1, followed by the REM state, before the non-REM state appears again. Each cycle may last for approximately 90-120 minutes for an adult. However, LF/HF bursts may occur in the REM state, resulting in a much higher LF/HF than in the non-REM state.

The disclosure may aim to provide an objective fatigue index based on HRV parameters. The LF/HF ratios from HRV data may be collected from a device with photoplethysmography (PPG) sensors or from a device with ECG sensors. The device with PPG sensors or ECG sensors may be a wearable device. The wearable device may be built to collect physiological signals with a pre-defined frequency and to calculate the LF/HF ratios. The measurement may be triggered periodically, e.g., per hour within 24 hours for 7 continuous days.

Physiological signals (e.g., HRV signals) may be obtained in a data collection process during an ECG measurement. Physiological signals may be recorded every 5 minutes up to 24 hours. HRV signals may be calculated as raw data of the recorded physiological signals. A device is used for overnight monitoring of ECG and HRV in cardiovascular assessments and studies. However, the device for ECG measurements may be designed for stationary rather than mobile measurements, which may be required in many physiological measurements. For calculating HRV parameters, the continuous inter-pulse intervals may be accumulated and converted into a wave, and R-R intervals (i.e., the intervals between two adjacent R waves) may be measured and averaged to obtain information regarding the LF and HF.

PPG may provide a noninvasive technology to detect physiological signals. PPG may use a light source and a photodetector at a subject's skin (a living body's skin) to measure the volumetric variations of blood circulation and monitor health conditions. Among the mobile designs of PPG, the wrist-worn design has become popular for calculating volumetric variations of blood circulation and in research that requires such raw data for ongoing analysis. PPG signals offer an excellent alternative to ECG recordings.

In the present disclosure, a wearable device (e.g., a smart band) with a PPG sensor detects physiological signals. Such wearable device collects PPG signals from individuals. The PPG signals may be converted into HRV parameters by the wearable device, a mobile device (e.g., a smart phone, a tablet, or a laptop) communicated with the wearable device, or a remote device (e.g., a cloud computing device or a fog computing device) communicated with the wearable device.

Each measurement for obtaining HRV parameters may require several minutes (e.g., 2 minutes) of detection, which may include obtaining heart rate and calculating several frequency-domain parameters (e.g., LF power and HF power) therefrom. The converted HF and LF information may be used to estimate sympathetic and parasympathetic activity to assess a subject's stress and fatigue.

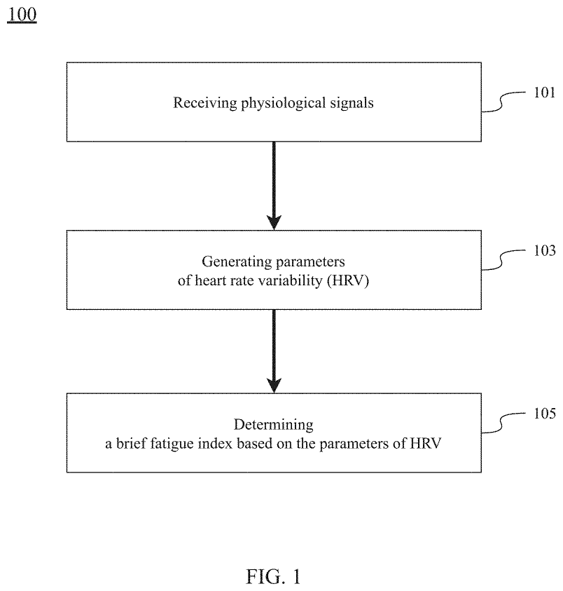

FIG. 1 is a flowchart of a process 100 in accordance with some embodiments of the present disclosure. Process 100 may determine a fatigue index (FI) in accordance with some embodiments of the present disclosure. In the present disclosure, an FI may be based on objective measurements on a subject (or a living body).

Process 100 may include operations 101, 103, and 105. In operation 101, physiological signals may be received or obtained, from, for example, a portable or wearable device. The physiological signals may be received from a device with ECG sensors. The physiological signals may be received from a device with PPG sensors. The physiological signals may be ECG signals. The physiological signals may be PPG signals. The PPG signals may include several waveform parameters, e.g., pulse amplitude and pulse slope. The physiological signals may be received within a period, which may be several minutes (e.g., 2 to 10 minutes), one or more hours (e.g., 1 to 5 hours), or one or more days.

In operation 103, parameters of heart rate variability (HRV) may be generated or calculated. Parameters of HRV may be generated or calculated from the physiological signals received or obtained in operation 101. The parameters of HRV may include RR intervals (i.e., the interval between two adjacent R waves). Upon processing the RR intervals, the parameters of HRV, including parameters in time domain, frequency domain, and time-frequency domain, may be obtained.

Parameters in time domain may include SDNN (Standard Deviation of Normal to Normal), SDANN (Standard Deviation of the Averages of NN intervals in all 5-minute segments of the entire recording), NN50 count (Number of pairs of adjacent NN intervals differing by more than 50 milliseconds in the entire recording), pNN50 (NN50 count divided by the total number of all NN intervals), and TINN (Triangular Interpolation of NN interval histogram). SDNN indicates the standard deviation of NN intervals (i.e., intervals of two normal heart beats), wherein the units are μs. For SDANN, the recording period is divided into consecutive segments of 5 minutes; the standard deviations of NN intervals for each consecutive segment of 5 minutes are calculated, and SDANN indicates the average of the standard deviations of NN intervals for the consecutive segments; the unit is μs. NN50 count indicates the number of pairs of adjacent NN intervals differing by more than 50 ms over the entire recording. HRV triangular index indicates the total number of all RR intervals divided by the height of the histogram of all RR intervals.

Parameters in frequency domain may be generated from RR intervals or NN intervals via Fourier transformation. Parameters in frequency domain may be represented as power spectral density or spectral distribution. Parameters of HRV in frequency domain can show the characteristics of 200 to 500 consecutive heart beats. The recording for generating parameters of HRV in frequency domain may last at least for several minutes (e.g., 2 to 10 minutes). The spectral distribution of RR intervals or NN intervals may be lower than 1 Hz. Some peaks may be found between 0 to 0.4 Hz. High frequency (HF) band of parameters of HRV in frequency domain may be ranged from 0.15 Hz to 0.4 Hz; low frequency (LF) band of parameters of HRV in frequency domain may be ranged from 0.04 Hz to 0.15 Hz. The power density or distribution in HF band may reflect the activity of the parasympathetic nerve. The power density or distribution in LF band may be controlled by the sympathetic nerve or the parasympathetic nerve.

Parameters of HRV in frequency domain may include total power (TP), very low frequency power (VLFP) (i.e., the power distributed in the frequency not exceeding 0.04 Hz), low frequency power (LFP), high frequency power (HFP), normalized LFP (nLFP) (i.e.,

$$\frac{LF P}{( TP - V LF P )} ) ,$$

normalized HFP (nHFP) (i.e.

$$\frac{HF P}{( TP - V LF P )} ) ,$$

and ratio of LFP to HFP

$$\frac{LF P}{HF P}$$

or LF/HF). LF/HF may indicate the balance of the activities of the autonomic nerve.

In operation 105, an FI may be based on objective measurements on a subject (or a living body). A fatigue index (FI) may be determined based on the parameters of HRV. An FI may be determined based on the parameters of HRV generated or calculated in operation 103. An FI may be determined based on the parameters of HRV in time domain. An FI may be determined based on the parameters of HRV in frequency domain. An FI may be determined based on the LF power and the HF power in frequency domain. An FI may be determined based on the ratio of the LF power to the HF power (LF/HF) in frequency domain. An FI may be determined based on the LF/HF ratios in different activity phases.

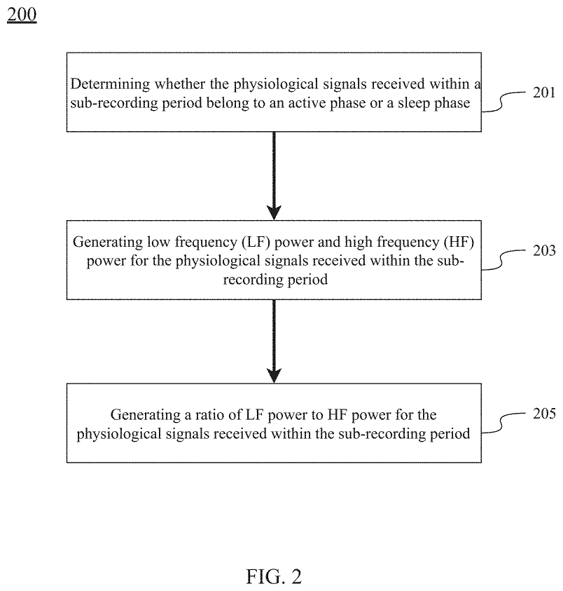

FIG. 2 is a flowchart of a process 200 in accordance with some embodiments of the present disclosure. Process 200 may generate or calculate parameters of heart rate variability (HRV) in accordance with some embodiments of the present disclosure.

Process 200 may include operations 201, 203, and 205. In operation 201, it may be determined to which phase the physiological signals belong. The physiological signals may be determined to belong to an active phase or a sleep phase (or a non-active phase). In some embodiments, daytime of a subject (or a living body) may include an active phase and a sleep phase (or a non-active phase).

The physiological signals used in operation 201 may be received in operation 101. The physiological signals used in operation 201 may be received within a sub-period. Such sub-period may be several minutes (e.g., 2 to 10 minutes) or several hours (e.g., 1 to 5 hours). An active phase may include one or more sub-periods. A sleep phase (or a non-active phase) may include one or more sub-periods. A period for recording physiological signals may include several sub-periods, several hours, one or more days, one or more active phases, or one or more sleep phases.

In operation 203, parameters in frequency domain may be generated or calculated based on the physiological signals received in the sub-period as used in operation 201. Parameters of HRV in frequency domain may be generated or calculated based on the physiological signals received in the sub-period. LF power and HF power may be generated or calculated based on the physiological signals received in the sub-period as used in operation 201.

In operation 205, a ratio of LF power to HF power (i.e., LF/IF) may be generated or calculated. LF/HF may be generated or calculated based on the LF power and HF power as generated in operation 203. LF/HF may be generated or calculated based on the physiological signals received in the sub-period as used in operation 201. After operation 205, a LF/HF ratio for a given sub-period (in an active phase or in a sleep phase) may be generated or calculated.

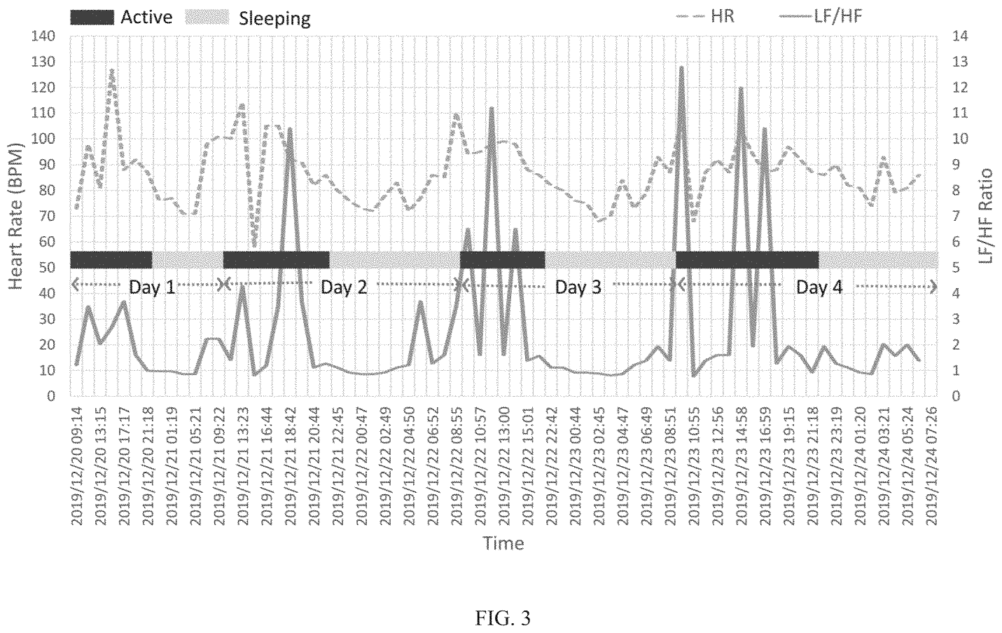

FIG. 3 shows curves of the heart rates and the LF/HF ratios in accordance with some embodiments of the present disclosure. In FIG. 3, the heart rates and the LF/HF ratios may be detected or measured from a subject (or living body). In FIG. 3, the period for recording the physiological signals may be 4 days. Each day in FIG. 3 includes an active phase and a sleep phase. The darker stripes indicate the active phases. The lighter stripes indicate the sleep phases.

The dotted line in FIG. 3 shows the heart rates (HR) (e.g., beats per minute) at different time points. The solid line in FIG. 3 shows the LF/HF ratios at different time points. A heart rate at one time point may be generated or calculated based on the physiological signal received in a sub-period; the sub-period may be several minutes or several hours. A LF/HF ratio at one time point may be generated or calculated based on the physiological signal received in a sub-period; the sub-period may be several minutes (e.g., 2 to 10 minutes) or several hours (e.g., 1 to 5 hours).

Typically, a high activity level may lead to an increased HR and increased LF/HF ratio. A higher average HR and a higher LF/HF ratio may be attained in an active phase. In the sleep phase, the HR may decrease gradually, and the LF/HF ratio may decrease from exceeding 1 to less than 1. A better sleep quality may be obtained if a decreased HR and a LF/HF ratio less than 1 are detected. Both the HR and the LF/HF ratio may increase at the end of the sleep phase.

The sleep event may also occur in the active phase of a patient. For example, low LF/IF ratios (i.e., less than 1) with low HRs may occur on Days 2 and 4. Moreover, the burst of the LF/IF ratio in the sleep phase of Day 2 may indicate an REM state in the sleep phase. HRV may be monitored in a timed-measurement random sampling manner, in which the HRV events may be randomly sampled in a pre-defined interval between two sub-periods. To precisely capture the target events, a fine-grained measurement interval between two sub-periods may be adopted in the timer configuration. Owing to the need for a large data storage space and electric power consumption, the sub-period for recording physiological signals may be set to 1 hour.

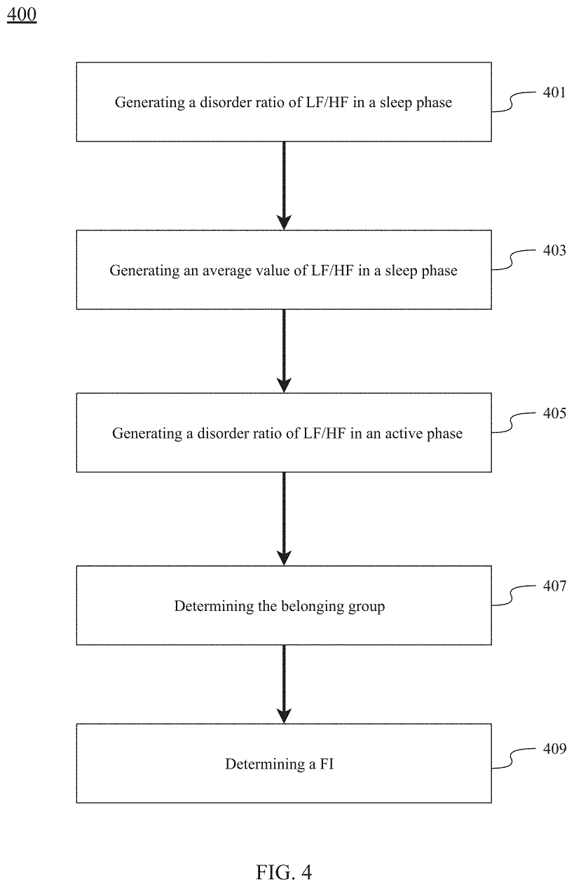

FIG. 4 is a flowchart of a process 400 in accordance with some embodiments of the present disclosure. Process 400 can determine an FI in accordance with some embodiments of the present disclosure.

Process 400 may include operations 401, 403, 405, 407, and 409. In operation 401, a disorder ratio of LF/HF in a sleep phase may be generated or calculated. A disorder ratio of LF/IF in a sleep phase may be represented as:

$${LHD}^{Sleep} = \frac{the number of \frac{LF}{HF} > BR in the sleep phase}{the number of \frac{LF}{HF} measured in the sleep phase} \qquad ( 1 )$$

The disorder ratio of LF/HF in a sleep phase may track the relationship between the sleep quality and fatigue. A higher value of LHD^{Sleep} may imply a shorter deep sleep duration.

In Formula (1) and the formula below, the variant “BR” is an abbreviation of “Balance Ratio.” In some embodiments, BR may be 1. In some embodiments, BR may be a LF/HF ratio in the non-REM state; BR may be a real number between 0.8 and 1.2 or between 0.5 and 1.5. In other embodiments, BR may be a LF/HF ratio in the REM state; BR may be a real number larger than 2. In some embodiments of the present disclosure, the variant “BR” may be used to check or determine the statuses or conditions in the deep sleep, and the variant “BR” may be selected to focus on the non-REM state. The selected value of BR may be associated with the gender and/or age of the subject. For example, the value of BR for an adult male may be configured to be 1.2. In another example, the value of BR for an adult female may be configured to be slightly smaller than 1. In some preferred embodiments of the present disclosure, the value of BR for a general adult may be configured to be 1.

In operation 403, an average value of LF/HF in a sleep phase may be generated or calculated. An average value of LF/HF in a sleep phase may be represented as:

$${LHA}^{Sleep} = \frac{\sum \frac{LF}{HF} measured in the sleep phase}{the number of \frac{LF}{HF} measured in the sleep phase} \qquad ( 2 )$$

In operation 405, a disorder ratio of LF/HF in an active phase may be generated or calculated. A disorder ratio of LF/HF in an active phase may be represented as:

$${LHD}^{Active} = \frac{the number of \frac{LF}{HF} < 1 in the active phase}{the number of \frac{LF}{HF} measured in the active phase} \qquad ( 3 )$$

The disorder ratio of LF/HF in an active phase may track the fatigue level during daytime rest. A higher value of LHD^{Active} may imply more frequent sleep events during the daytime. In Formula (3), the variant “BR” is an abbreviation of “Balance Ratio”. In some embodiments, BR may be 1. BR may be a real number between 0.8 and 1.2 or between 0.5 and 1.5.

In operation 407, the belonging group may be determined. It may be determined which group the physiological signals received in a period (e.g., including several hours or at least one day; including at least one active phase and at least one sleep phase) belongs. The physiological signals received in a period (e.g., including several hours or at least one day; including at least one active phase and at least one sleep phase) may be determined to belong to one of several groups (e.g., two groups, three groups, four groups, etc.). For example, the physiological signals received in a period may be determined to belong to one of Group A, Group B, and Group C. In some embodiments, the physiological signals received in a period may be determined to belong to one of Group A, Group B, Group C, and Group D.

In operation 407, the belonging group may be determined based on the physiological signals received in a period (e.g., including several hours or at least one day; including at least one active phase and at least one sleep phase). The belonging group may be determined based on the parameters of HRV generated or calculated from the physiological signals received in a period. The belonging group may be determined based on the LF/HF ratios generated or calculated from the physiological signals received in a period. The belonging group may be determined based on the LHD^{Sleep}, LHA^{Sleep}, and LHD^{Active} generated or calculated from the physiological signals received in a period.

Operation 407 may include determining the belonging group of the generated LHD^{Sleep} (i.e., Group^{LHDS}) The generated LHD^{Sleep} may be determined to belong to one of several groups (e.g., one of Group A, Group B, and Group C; Group^{LHDS}∈{A, B, C}). For example, the generated LHD^{Sleep} may be determined to belong to Group A when the generated LHD^{Sleep} is lower than the threshold TH₁^{LHDS}; the generated LHD^{Sleep} may be determined to belong to Group B when the generated LHD^{Sleep} is exceeding the threshold TH₁^{LHDS} and lower than the threshold TH₂^{LHDS}; and the generated LHD^{Sleep} may be determined to belong to Group C when the generated LHD^{Sleep} is exceeding the threshold TH₂^{LHDS}. The determination of the belonging grouped of the generated LHD^{Sleep} may be represented as:

$$\{ \begin{matrix}{Group}^{LHDS} = A \vert {LHD}^{Sleep} \leq {TH}_{1}^{LHDS} \\ {Group}^{LHDS} = B \vert {TH}_{1}^{LHDS} < {LHD}^{Sleep} \leq {TH}_{2}^{LHDS} \\ {Group}^{LHDS} = C \vert {LHD}^{Sleep} > {TH}_{2}^{LHDS}\end{matrix} \qquad ( 4 )$$

Operation 407 may include determining the belonging group of the generated LHA^{Sleep} (i.e., Group^{LHAS}) The generated LHA^{Sleep} may be determined to belong to one of several groups (e.g., one of Group A, Group B, and Group C; Group^{LHDS}∈{A, B, C}). For example, the generated LHA^{Sleep} may be determined to belong to Group A when the generated LHA^{Sleep} is lower than the threshold TH₁^{LHAS}; the generated LHA^{Sleep} may be determined to belong to Group B when the generated LHA^{Sleep} is exceeding the threshold TH₁^{LHAS} and lower than the threshold TH₂^{LHAS}; and the generated LHA^{Sleep} may be determined to belong to Group C when the generated LHA^{Sleep} is exceeding the threshold TH₂^{LHAS}. The determination of the belonging grouped of the generated LHA^{Sleep} may be represented as:

$$\{ \begin{matrix}{Group}^{LHAS} = A \vert {LHA}^{Sleep} \leq {TH}_{1}^{LHAS} \\ {Group}^{LHAS} = B \vert {TH}_{1}^{LHAS} < {LHA}^{Sleep} \leq {TH}_{2}^{LHAS} \\ {Group}^{LHAS} = C \vert {LHA}^{Sleep} > {TH}_{2}^{LHAS}\end{matrix} \qquad ( 5 )$$

Operation 407 may include determining the belonging group of the generated LHD^{Active} (i.e., Group^{LHDA}). The generated LHD^{Active} may be determined to belong to one of several groups (e.g., one of Group A, Group B, and Group C; Group^{LHDS}∈{A, B, C}). For example, the generated LHD^{Active} may be determined to belong to Group A when the generated LHD^{Active} is lower than the threshold TH₁^{LHDA}; the generated LHD^{Active} may be determined to belong to Group B when the generated LHD^{Active} exceeds the threshold TH₁^{LHDA} and lower than the threshold TH₂^{LHDA}; and the generated LHD^{Active} may be determined to belong to Group C when the generated LHD^{Active} exceeds the threshold TH₂^{LHDA}. The determination of the belonging grouped of the generated LHD^{Active} may be represented as:

$$\{ \begin{matrix}{Group}^{LHDA} = A \vert {LHD}^{Active} \leq {TH}_{1}^{LHDA} \\ {Group}^{LHDA} = B \vert {TH}_{1}^{LHDA} < {LHD}^{Active} \leq {TH}_{2}^{LHDA} \\ {Group}^{LHDA} = C \vert {LHD}^{Active} > {TH}_{2}^{LHDA}\end{matrix} \qquad ( 6 )$$

Operation 407 may include determining the belonging group of the physiological signals received in a period (e.g., including several hours or at least one day; including at least one active phase and at least one sleep phase) based on the belonging group of the generated LHD^{Sleep}, the belonging group of the generated LHA^{Sleep}, and the belonging group of the generated LHD^{Active} (i.e., Group^{LHDS}, Group^{LHAS}, and Group^{LHDA}).

The belonging group of the physiological signals received in a period (i.e., Group^{PS}) may be determined based on the majority of the belonging group of the generated LHD^{Sleep} the belonging group of the generated LHA^{Sleep}, and the belonging group of the generated LHD^{Active} (i.e., Group^{LHDS}, Group^{LHAS}, and Group^{LHDA}). In some embodiments, Group^{PS}, Group^{LHDS}, Group^{LHAS}, Group^{LHDA}∈{A, B, C}. For example, when Group^{LHDS}=A, Group^{LHAS}=A, and Group^{LHDA}=A, then Group^{PS}=A. When Group^{LHDS}=B, Group^{LHAS}=B, and Group^{LHDA}=C, then Group^{PS}=B. When Group^{LHDS}=B Group^{LHAS}=C, and Group^{LHDA}=C, then Group^{PS}=C.

The belonging group of the physiological signals received in a period may be determined based on weighted values of the belonging group of the generated LHD^{Sleep}, the belonging group of the generated LHA^{Sleep}, and the belonging group of the generated LHD^{Active}. For example, Group^{PS} may be determined based on W₁Group^{LHDS}+W₂Group^{LHAS}+W₃Group^{LHDA}. Group^{PS} represents the belonging group of the received physiological signals; Group^{LHDS} represents the belonging group of LHD^{Sleep}; Group^{LHAS} represents the belonging group of LHA^{Sleep}; Group^{LHDA} represents the belonging group of LHD^{Active}; W₁, W₂, and W₃ are weighting values; and Group^{PS}, Group^{LHDS}, Group^{LHAS}, Group^{LHDA}∈{A, B, C}.

In some embodiments, W₁, W₂, W₃∈{ [symbol] |W₁+W₂+W₃=1}. The determination of Group^{PS} may be represented as:

$$\{ \begin{matrix}{Group}^{PS} = A \vert {W}_{1} {Group}^{LHDS} + {W}_{2} {Group}^{LHAS} + {W}_{3} {Group}^{LHDA} > 0.5 A \\ {Group}^{PS} = B \vert {W}_{1} {Group}^{LHDS} + {W}_{2} {Group}^{LHAS} + {W}_{3} {Group}^{LHDA} > 0.5 B \\ {Group}^{PS} = C \vert {W}_{1} {Group}^{LHDS} + {W}_{2} {Group}^{LHAS} + {W}_{3} {Group}^{LHDA} > 0.5 C\end{matrix} \qquad ( 7 )$$

For example, W₁ may be 0.52, W₂ may be 0.26, and W₃ may be 0.22, In this case, the priority of Group^{LHDS} is higher than the other two, and the priority of Group^{LHAS} is higher than Group^{LHDA}. In this example, when Group^{LHDS} is A, Group^{LHAS} is B, and Group^{LHDA} is C, Group^{PS} may be A. In the example, when Group^{LHDS} is B and Group^{LHAS} and Group^{LHDA} are C, Group^{PS} is B.

In some embodiments, when W₁ is configured to be much larger than W₂ and W₃, the Group^{PS} may be determined based on Group^{LHDS}. For example, when W₁ is configured to be 0.8 (which may be much larger than W₂ and W₃), the Group^{PS} may be determined based on Group^{LHDS} in the case that the Group^{LHDS}, Group^{LHAS}, and Group^{LHDA} are different. In some examples, the Group^{PS} may be determined merely based on Group^{LHDS}.

In operation 409, an FI may be determined, based, for example, on the physiological signals received in a period (e.g., including several hours or at least one day; including at least one active phase and at least one sleep phase). An FI may be determined based on the parameters of HRV generated or calculated from the physiological signals received in a period. An FI may be determined based on the LF/HF ratios generated or calculated from the physiological signals received in a period. An FI may be determined based on the LHD^{Sleep}, LHA^{Sleep}, and LHD^{Active} generated or calculated from the physiological signals received in a period. An FI may be determined based on the belonging group of the received physiological signals.

The FI may be determined via the formula as follows.

$$\{ \begin{matrix}FI = {α}_{A} {LHD}^{Sleeping} + {β}_{A} {LHA}^{Sleeping} + {γ}_{A} {LHD}^{Active} + {ɛ}_{A} \vert {Group}^{PS} = A \\ FI = {α}_{B} {LHD}^{Sleeping} + {β}_{B} {LHA}^{Sleeping} + {γ}_{B} {LHD}^{Active} + {ɛ}_{B} \vert {Group}^{PS} = B \\ FI = {α}_{C} {LHD}^{Sleeping} + {β}_{C} {LHA}^{Sleeping} + {γ}_{C} {LHD}^{Active} + {ɛ}_{C} \vert {Group}^{PS} = C\end{matrix} \\ {α}_{A} , {β}_{A} , {γ}_{A} , {ɛ}_{A} , {α}_{B} , {β}_{B} , {γ}_{B} , {ɛ}_{B} , {α}_{C} , {β}_{C} , {γ}_{C} , {ɛ}_{C} ∈ ℝ \qquad ( B )$$

Weighting factors α_{A}, β_{A}, γ_{A}, α_{B}, β_{B}, γB, α_{C}, β_{C}, γ_{C} may be employed to amplify or reduce the components of the corresponding HRV parameters. The weighting factors may depend on their correlation with FI and their respective value range contributed to FI. When fitting Formula (8) into a multiple linear regression model, the shifting factors ε_{A}, ε_{B}, ε_{C} may correspond to the intercept of the model and may be based on the available dataset. In some embodiments, all of the weighting factors α_{A}, β_{A}, γ_{A}, α_{B}, β_{B}, γB, α_{C}, β_{C}, γ_{C} may be 1.

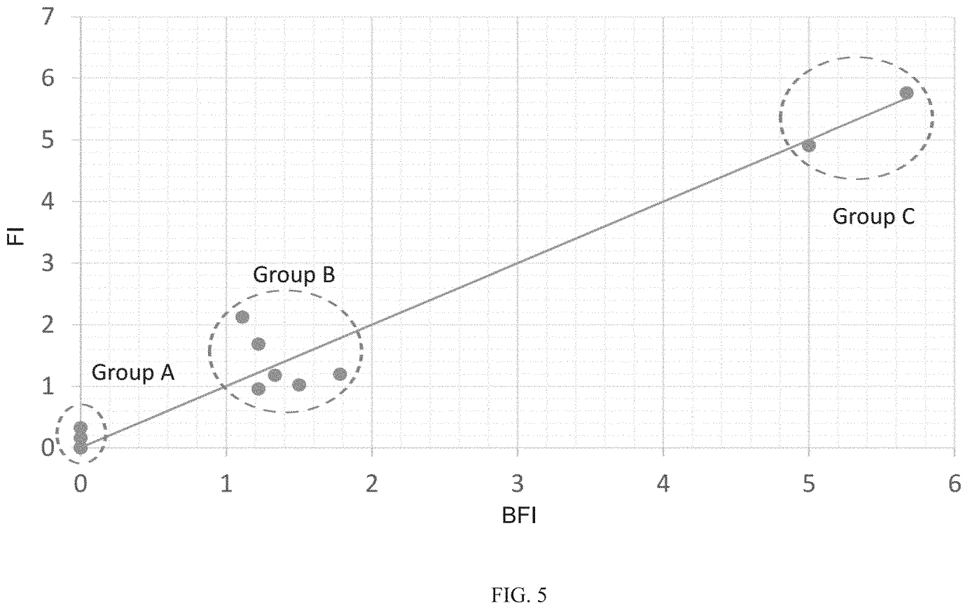

FIG. 5 depicts the relationship between the BFI (e.g., an index based on subjective experiences) and the FI (e.g., an index based on objective measurements) in accordance with some embodiments of the present disclosure. FIG. 5 shows the distribution between the BFI and the FI in accordance with some embodiments of the present disclosure. The BFI shown in FIG. 5 may be obtained from questionnaires. The FI shown in FIG. 5 may be obtained or generated from one or more operations shown in FIGS. 1, 2, and 4 in accordance with some embodiments of the present disclosure. FIG. 5 shows that the FI obtained in accordance with some embodiments of the present disclosure and the subjective obtained from questionnaires may be close.

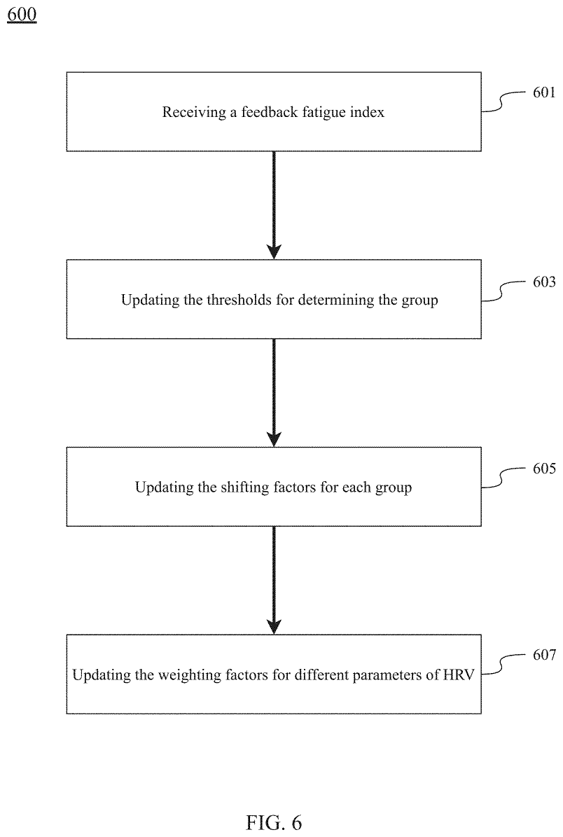

FIG. 6 is a flowchart of a process 600 in accordance with some embodiments of the present disclosure. Process 600 can update factors in accordance with some embodiments of the present disclosure. Process 600 can update one or more of the weighting factors α_{A}, β_{A}, γ_{A}, α_{B}, β_{B}, γB, α_{C}, β_{C}, γ_{C}. Process 600 can update one or more of the shifting factors ε_{A}, ε_{B}, ε_{C}. Process 600 can update one or more of the thresholds TH₁^{LHDS}, TH₂^{LHDS}, TH₁^{LHAS}, TH₂^{LHAS}, TH₁^{LHDA}, TH₂^{LHDA}, which are used to determine Group^{LHDS}, Group^{LHAS}, Group^{LHDA}. Process 600 can update one or more of the weighting values W₁, W₂, W₃, which can determine Group^{PS}.

Process 600 may include operations 601, 603, 605, and 607. In operation 601, a feedback fatigue index may be received. The feedback fatigue index may be input by the user. The feedback fatigue index may be input through a computing device. The feedback fatigue index may be input through a portable device (e.g., a smart phone or a laptop). The feedback fatigue index may be input through a wearable device (e.g., a smart band). The feedback fatigue index may be determined based on a questionnaire including several questions. The feedback fatigue index may be based on the subjective experiences of the user.

In operation 603, the thresholds for determining the belong group may be updated. One or more of the weighting values W₁, W₂, W₃, which may be used to determine Group^{PS}, may be modified or updated. One or more of the thresholds TH₁^{LHDS}, TH₂^{LHDS}, TH₁^{LHAS}, TH₂^{LHAS}, TH₁^{LHDA}, TH₂^{LHDA}, which may be used to determine Group^{LHDS}, Group^{LHAS}, Group^{LHDA}, may be modified or updated.

The update of operation 603 may be based on the difference between the FI and the feedback fatigue index. The update of operation 603 may be based on the distance(s) between the belonging group and one or two adjacent groups.

In operation 605, one or more shifting factors may be updated. One or more of the shifting factors ε_{A}, ε_{B}, ε_{C} may be modified or updated. In some embodiments, only the shifting factor of the belonging group may be modified or updated. For example, when Group^{PS}=B, only the shifting factor ε_{B} may be modified or updated. The update of operation 605 may be based on the difference between the FI and the feedback fatigue index.

In operation 607, one or more weighting factors may be updated. One or more of the weighting factors α_{A}, β_{A}, γ_{A}, α_{B}, β_{B}, γB, α_{C}, β_{C}, γ_{C} may be modified or updated. In some embodiments, only the weighting factors of the belonging group may be modified or updated. For example, when Group^{PS}=C, only the weighting factors α_{C}, β_{C}, γ_{C} may be modified or updated. The update of operation 607 may be based on the difference between the FI and the feedback fatigue index. The update of operation 607 may be based on the distance(s) between the belonging group and one or two adjacent groups.

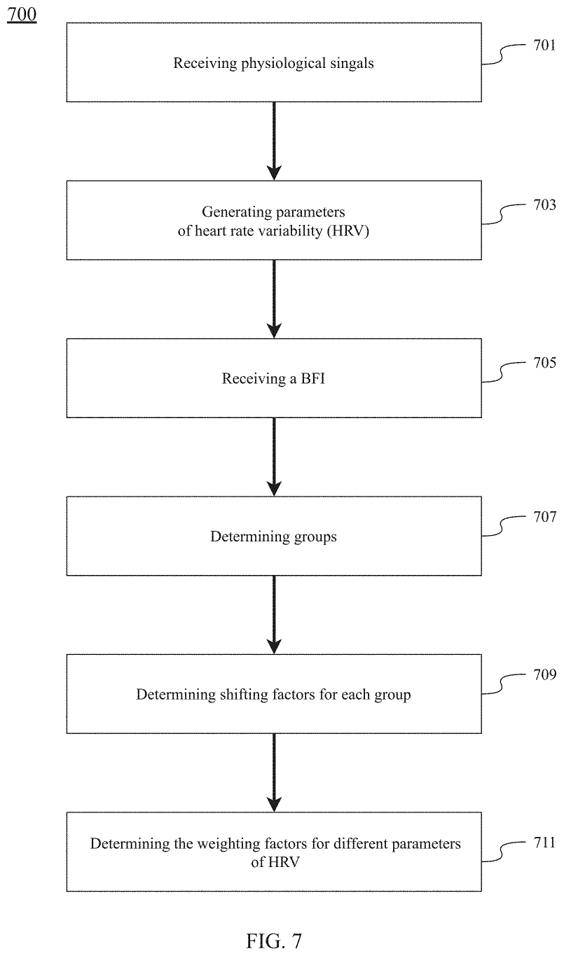

FIG. 7 is a flowchart of a process 700 in accordance with some embodiments of the present disclosure. Process 700 may determine factors in accordance with some embodiments of the present disclosure. Process 700 may determine weighting factors and shifting factors.

Process 700 may include operations 701, 703, 705, 707, 709, and 711. In operation 701, physiological signals may be received or obtained. The physiological may be received from a portable device or a wearable device. The physiological signals may be received from a device with ECG sensors. The physiological signals may be received from a device with PPG sensors. The physiological signals may be ECG signals. The physiological signals may be PPG signals. The PPG signals may include several waveform parameters, e.g., pulse amplitude and pulse slope. The physiological signals may be received within a period, which may be several hours or several days. The physiological signals for several subjects undergoing testing (or several living bodies undergoing testing) may be received or detected within a period.

In operation 703, parameters of heart rate variability (HRV) may be generated or calculated. Parameters of HRV may be generated or calculated from the physiological signals received or obtained in operation 101. The parameters of HRV may include RR intervals (i.e., the intervals between adjacent two R waves). Upon processing the RR intervals, the parameters of HRV may include parameters in time domain, frequency domain, and time-frequency domain. In some embodiments, a disorder ratio of LF/HF in a sleep phase LHD^{Sleep}, an average value of LF/HF in a sleep phase LHA^{Sleep}, and a disorder ratio of LF/HF in an active phase LHD^{Active} for each subject undergoing testing may be generated or calculated in operation 703.

In operation 705, a BFI (e.g., a subjective index) may be received. The BFI may be input by the user. The BFI may be input through a computing device. The BFI may be input through a portable device (e.g., a smart phone or a laptop). The BFI may be input through a wearable device (e.g., a smart band). The BFI may be determined based on a questionnaire including several questions. The BFI for each subject undergoing testing may be received.

In operation 707, the belonging group for each subject under may be determined. It may be determined for each subject undergoing testing which group the physiological signals received in a period (e.g., including several hours or at least one day; including at least one active phase and at least one sleep phase) belongs. The physiological signals received in a period for each subject undergoing testing may be determined to belong to one of several groups (e.g., one of Group A, Group B, and Group C).

In operation 707, the belonging group may be determined based on the physiological signals received in a period for each subject undergoing testing. For each subject undergoing testing, the belonging group may be determined based on the parameters of HRV generated or calculated from the physiological signals received in a period. For each subject undergoing testing, the belonging group may be determined based on the LF/HF ratios generated or calculated from the physiological signals received in a period. For each subject undergoing testing, the belonging group may be determined based on the LHD^{Sleep}, LHA^{Sleep}, and LHD^{Active} generated or calculated from the physiological signals received in a period.

Operation 707 may include determining the belonging group of the generated LHD^{Sleep} (i.e., Group^{LHDS}) for each subject undergoing testing. For each subject undergoing testing, the generated LHD^{Sleep} may be determined to belong to one of several groups (e.g., one of Group A, Group B, and Group C; Group^{LHDS}∈{A, B, C}). The determination of the belonging grouped of the generated LHD^{Sleep} for each subject undergoing testing may be represented as with Formula (4).

Operation 707 may include determining the belonging group of the generated LHA^{Sleep} (i.e., Group^{LHAS}) for each subject undergoing testing. For each subject undergoing testing, the generated LHA^{Sleep} may be determined to belong to one of several groups (e.g., one of Group A, Group B, and Group C; Group^{LHAS}∈{A, B, C}). The determination of the belonging grouped of the generated LHA^{Sleep} for each subject undergoing testing may be represented as in Formula (5).

Operation 707 may include determining the belonging group of the generated LHD^{Active} (i.e., Group^{LHDA}) for each subject undergoing testing. For each subject undergoing testing, the generated LHD^{Active} may be determined to belong to one of several groups (e.g., one of Group A, Group B, and Group C; Group^{LHDA}∈{A, B, C}). The determination of the belonging group of the generated LHD^{Active} for each subject undergoing testing may be represented as in Formula (6).

Operation 707 may include determining the belonging group of the physiological signals received in a period (e.g., including several hours or at least one day; including at least one active phase and at least one sleep phase) (i.e., Group^{PS}), for each subject undergoing testing, based on the belonging group of the generated LHD^{Sleep}, the belonging group of the generated LHA^{Sleep}, and the belonging group of the generated LHD^{Active} (i.e., Group^{LHDS}, Group^{LHAS}, and Group^{LHDA}).

For each subject undergoing testing, the belonging group of the physiological signals received in a period (i.e., Group^{PS}) may be determined based on the majority of the belonging group of the generated LHD^{Sleep}, the belonging group of the generated LHA^{Sleep}, and the belonging group of the generated LHD^{Active} (i.e., Group^{LHDS}, Group^{LHAS}, and Group^{LHDA}).

For each subject undergoing testing, the belonging group of the physiological signals received in a period may be determined based on weighted values of the belonging group of the generated LHD^{Sleep}, the belonging group of the generated LHA^{Sleep}, and the belonging group of the generated LHD^{Active}. The determination of the belonging group of the physiological signals received in a period for each subject undergoing testing may be represented as in Formula (7).

In operation 709, the shifting factors for each group may be determined. In some embodiments, the shifting factor ε_{A} for Group A may be determined based on the subjects undergoing testing whose belonging group is Group A (i.e., Group^{PS}=A). The shifting factor ε_{B} for Group B may be determined based on the subjects undergoing testing whose belonging group is Group B (i.e., Group^{PS}=B). The shifting factor ε_{C} for Group C may be determined based on the subjects undergoing testing whose belonging group is Group C (i.e., Group^{PS}=C). The determination of the shifting factors may be based on the difference between the FI and the BFI. The shifting factors may be determined wherein all of the weighting factors α_{A}, β_{A}, γ_{A}, α_{B}, β_{B}, γ_{B}, α_{C}, β_{C}, γ_{C} are set to 1.

In operation 711, the weighting factors for each group may be determined. In some embodiments, the weighting factors α_{A}, β_{A}, γ_{A} for Group A may be determined based on the subjects undergoing testing whose belonging group is Group A (i.e., Group^{PS}=A). The weighting factors α_{B}, β_{B}, γ_{B} for Group B may be determined based on the subjects undergoing testing whose belonging group is Group B (i.e., Group^{PS}=B). The weighting factors α_{C}, β_{C}, γ_{C} for Group C may be determined based on the subjects undergoing testing whose belonging group is Group C (i.e., Group^{PS}=C). The determination of weighting factors may be based on the difference between the FI and the feedback fatigue index. The determination of weighting factors may be based on the distance(s) between the belonging group and one or two adjacent groups.

After process 700, the initial shifting factors ε_{A}, ε_{B}, ε_{C} and the initial weighting factors α_{A}, β_{A}, γ_{A}, α_{B}, β_{B}, γ_{B}, α_{C}, β_{C}, γ_{C} may be determined for generating or calculating the FI.

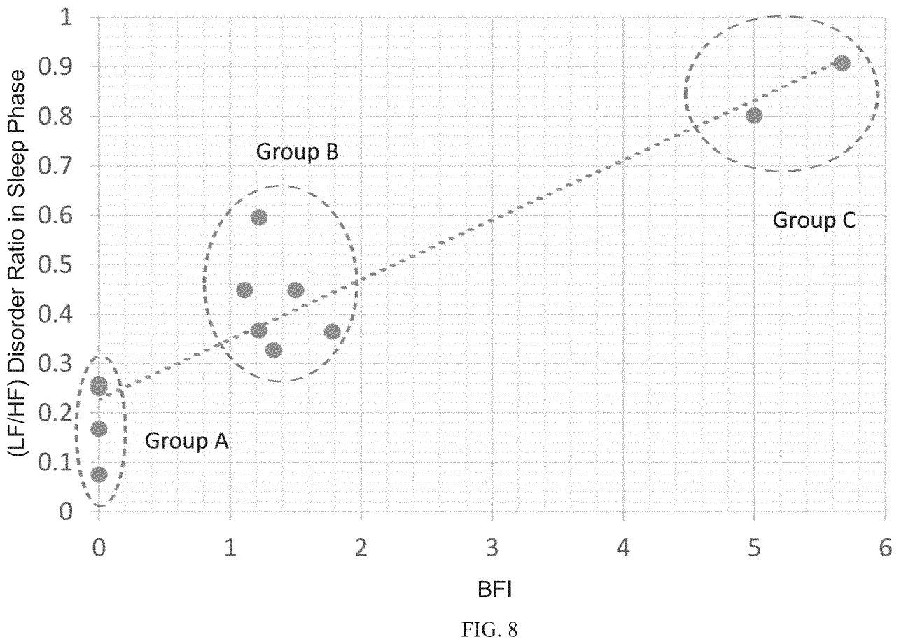

FIG. 8 depicts the relationship between the BFI (e.g., a subjective BFI) and the LF/IF disorder ratios in the sleep phase in accordance with some embodiments of the present disclosure. The BFI may be determined based on a questionnaire including several questions. A datapoint in FIG. 8 may correspond to a subject undergoing testing. A datapoint in FIG. 8 may correspond to a subject undergoing testing within a period (e.g., an entire recording period). In FIG. 8, the datapoints may be grouped into three groups (e.g., Group A, Group B, and Group C). FIG. 8 shows that: (1) the LF/HF disorder ratio increases with BFI in the sleep phase; (2) and that a positive correlation between the two axes (ρ=0.93); and (3) three distinct BFI groups, namely Groups A (BFI=0), B (BFI arounds 1 to 2), and C (BFI>3), exists between LF/HF disorder ratio of 0 to 1.

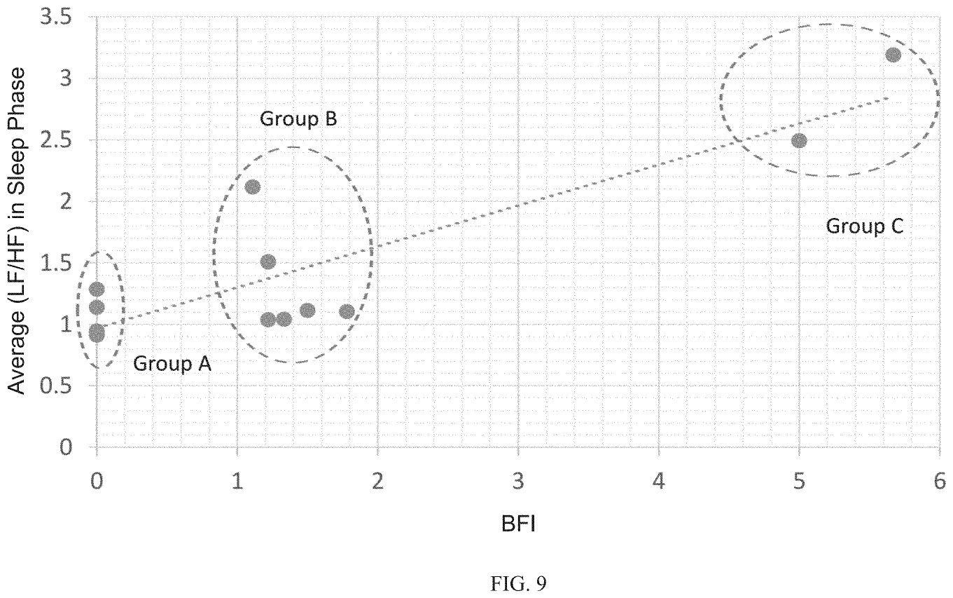

FIG. 9 depicts the relationship between the BFI (e.g., a subjective BFI) and the average values of LF/HF ratios in the sleep phase in accordance with some embodiments of the present disclosure. The BFI may be determined based on a questionnaire including several questions. A datapoint in FIG. 9 may correspond to a subject undergoing testing. A datapoint in FIG. 9 may correspond to a subject undergoing testing within a period (e.g., an entire recording period). In FIG. 9, the datapoints may be grouped into three groups (e.g., Group A, Group B, and Group C). FIG. 9 may show a positive correlation between the average LF/HF and BFI in the sleep phase (correlation coefficient ρ=0.86).

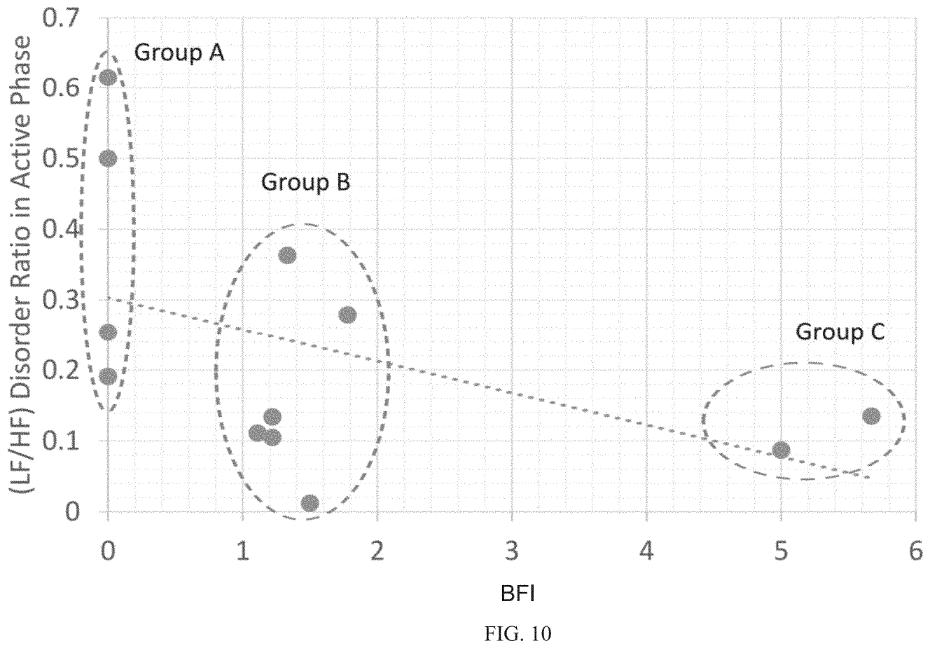

FIG. 10 depicts the relationship between the BFI (e.g., a subjective BFI) and the LF/HF disorder ratios in the active phase in accordance with some embodiments of the present disclosure. The BFI may be determined based on a questionnaire including several questions. A datapoint in FIG. 10 may correspond to a subject undergoing testing. A datapoint in FIG. 10 may correspond to a subject undergoing testing within a period (e.g., an entire recording period). In FIG. 10, the datapoints may be grouped into three groups (e.g., Group A, Group B, and Group C). FIG. 10 may show a negative correlation in the active phase (correlation coefficient ρ=−0.47).

From FIGS. 8 and 9, sleep quality may strongly relate to the fatigue or the BFI (e.g., a subjective index). From FIGS. 8-10, subjects with high BFI (e.g., high fatigue level) may have poor sleep quality at night and insufficient daytime rest. The sleep quality of subjects with lower BFI (e.g., lower fatigue level) may be generally better at night, and the resting frequency at daytime varies.

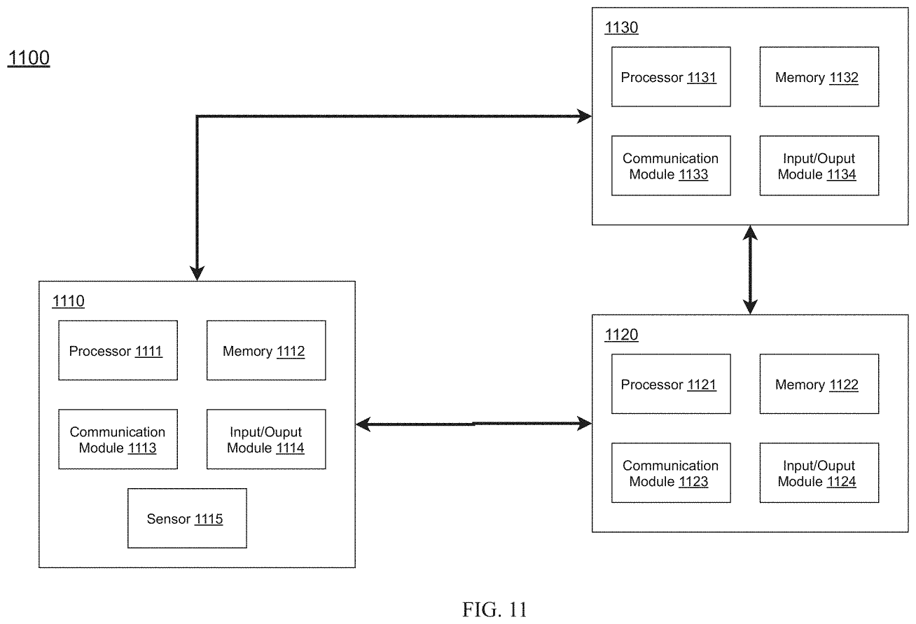

FIG. 11 is a diagram of a system 1100 in accordance with some embodiments of the present disclosure. System 1100 may include a wearable device 1110 (e.g., a smart band), a portable device 1120 (e.g., a smart phone or a laptop), and a computing device (e.g., a cloud server or a fog server).

The wearable device 1110 may include a processor 1111, a memory 1112, a communication module 1113, an input/output module 1114, and at least one sensor 1115 coupled with each other. The memory 1112 may be a non-transitory computer-readable medium. The memory 1112 may include computer executable programs wherein, when the processor 1111 executes the programs, the wearable device 1110 and its components may be directed to perform one or more operations of processes 100, 200, 400, 600, and 700.

The sensor 1115 may collect or detect physiological signals from a subject or a living body. The sensor 1115 may be an ECG sensor or a PPG sensor. The processor 1111 may be caused by programs to receive physiological signals from the sensor 1115.

The communication module 1113 may communicate with the portable device 1120 and the computing device 1130. The communication protocol between the wearable device 1110 and the portable device 1120 may be a wired protocol or a wireless protocol. The communication protocol between the wearable device 1110 and the computing device 1130 may be a wired protocol or a wireless protocol. The wired protocol may include Universal Serial Bus (USB). The wireless protocol may include Bluetooth (e.g., Bluetooth Low Energy), IEEE 802.11 (e.g., Wi-Fi 4, Wi-Fi 5, or Wi-Fi 6), 3GPP Long-Term Evolution (LTE) (4G), and 3GPP New Radio (5G).

The portable device 1120 may include a processor 1121, a memory 1122, a communication module 1123, and an input/output module 1124 coupled with each other. The memory 1122 may be a non-transitory computer-readable medium. The memory 1122 may include computer executable programs wherein when the processor 1121 executes the programs, the portable device 1120 and its components may be directed to perform one or more operations of processes 100, 200, 400, 600, and 700.

The communication module 1123 may communicate with the portable device 1110 and the computing device 1130. The communication protocol between the wearable device 1110 and the portable device 1120 may be a wired protocol or a wireless protocol. The communication protocol between the portable device 1120 and the computing device 1130 may be a wired protocol or a wireless protocol. The wired protocol may include Universal Serial Bus (USB). The wireless protocol may include Bluetooth (e.g., Bluetooth Low Energy), IEEE 802.11 (e.g., Wi-Fi 4, Wi-Fi 5, or Wi-Fi 6), 3GPP Long-Term Evolution (LTE) (4G), and 3GPP New Radio (5G).

The computing device 1130 may include a processor 1131, a memory 1132, a communication module 1133, and an input/output module 1134 coupled with each other. The memory 1132 may be a non-transitory computer-readable medium. The memory 1132 may include computer executable programs wherein, when the processor 1131 executes the programs, the computing device 1130 and its components may be directed to perform one or more operations of processes 100, 200, 400, 600, and 700.

The communication module 1133 may communicate with the portable device 1110 and the portable device 1120. The communication protocol between the wearable device 1110 and computing device 1130 may be a wired protocol or a wireless protocol. The communication protocol between the portable device 1120 and the computing device 1130 may be a wired protocol or a wireless protocol. The wired protocol may include Universal Serial Bus (USB). The wireless protocol may include Bluetooth (e.g., Bluetooth Low Energy), IEEE 802.11 (e.g., Wi-Fi 4, Wi-Fi 5, or Wi-Fi 6), 3GPP Long-Term Evolution (LTE) (4G), and 3GPP New Radio (5G).

Accordingly, the operations of generating parameters of HRV may be performed on at least one of the wearable device 1110, the portable device 1120, and the computing device 1130. The operations of determining an FI (e.g., an index based on objective measurements) may be performed on at least one of the wearable device 1110, the portable device 1120, and the computing device 1130. The operations of updating weighting factors, shifting factors, and/or thresholds for groups may be performed on at least one of the wearable device 1110, the portable device 1120, and the computing device 1130. The operations of determining initial weighting factors, initial shifting factors, and/or initial thresholds for groups may be performed on at least one of the wearable device 1110, the portable device 1120, and the computing device 1130.

In some embodiments, the present disclosure provides a method of determining a fatigue index. The method may include receiving physiological signals; generating a plurality of parameters of heart rate variability based on the physiological signals; and determining the fatigue index based on the plurality of parameters of heart rate variability.

In some embodiments, the present disclosure provides an apparatus. The apparatus may include at least one memory having computer executable instructions stored therein; and at least one processor coupled to the at least one memory. The computer executable instructions may cause the at least one processor to perform operations. The operations may include receiving physiological signals; generating a plurality of parameters of heart rate variability based on the physiological signals; and determining the fatigue index based on the plurality of parameters of heart rate variability.

The scope of the present application is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed, that perform substantially the same function or achieve substantially the same result as the corresponding embodiments described herein may be utilized according to the present disclosure. Accordingly, the appended claims are intended to include within their scope such as processes, machines, manufacture, compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of this application.

An alternative embodiment preferably implements the methods, processes, or operations according to embodiments of the present disclosure in a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein. The computer programmable instructions are configured to implement a method for emotion recognition from speech as stated above or other method according to an embodiment of the present disclosure.

While this application has been described with specific embodiments thereof, it is evident that many alternatives, modifications, and variations may be apparent to those skilled in the art. For example, various components of the embodiments may be interchanged, added, or substituted in the other embodiments. Also, all the elements of each figure are not necessary for operation of the disclosed embodiments. For example, one of ordinary skill in the art of the disclosed embodiments would be enabled to make and use the teachings of the application by simply employing the elements of the independent claims. Accordingly, embodiments of the application as set forth herein are intended to be illustrative, not limiting. Various changes may be made without departing from the spirit and scope of the application.

What is claimed is:

1. A method of determining a fatigue index, comprising:
receiving physiological signals;
generating a plurality of parameters of heart rate variability based on the physiological signals; and
determining the fatigue index based on the plurality of parameters of heart rate variability.

2. The method of claim 1, further comprising:
determining whether the physiological signals received in a first time period are obtained from a first phase or a second phase;
generating a low frequency (LF) power and a high frequency (HF) power for physiological signals received in the first time period; and
generating a ratio of the LF power to the HF power.

3. The method of claim 2, further comprising:
generating a first disorder ratio of the ratio of the LF power to the HF power in a first phase;
generating a first average value of the ratio of the LF power to the HF power in a first phase; and
generating a second disorder ratio of the ratio of the LF power to the HF power in a second phase,
wherein the first phase and the second phase include multiple first time periods.

4. The method of claim 3, wherein:
the first disorder ratio is a percentage that the ratio of the LF power to the HF power is greater than a balance ratio, and
the second disorder ratio is a percentage that the ratio of the LF power to the HF power is smaller than a balance ratio.

5. The method of claim 3, further comprising:
determining a belonging group of the received physiological signals received in a second time period, wherein the second time period includes the first phase and the second phase; and
determining a fatigue index based on the belonging group of the received physiological signals.

6. The method of claim 5, further comprising:
determining a first sub-group based on the first disorder ratio;
determining a second sub-group based on the first average value;
determining a third sub-group based on the second disorder ratio; and
determining, based on the first, second, and third sub-groups, the belonging group of the received physiological signals received in the second time period.

7. The method of claim 6, wherein the belonging group of the received physiological signals received in the second time period is determined based on the majority of the first, second, and third sub-groups.

8. The method of claim 6, wherein when the first, second, and third sub-groups are different, the belonging group of the received physiological signals received in the second time period is determined based on the first sub-group.

9. The method of claim 5, wherein the fatigue index is determined based on the first disorder ratio, the first average value, the second disorder ratio, and a shifting value the belonging group.

10. The method of claim 9, further comprising:
receiving a feedback fatigue index;
adjusting a threshold of determining the belonging group of the received physiological signals received in the second time period; and
adjusting the shifting value of the belonging group.

11. An apparatus, comprising:
at least one memory having computer executable instructions stored therein; and
at least one processor coupled to the at least one memory,
wherein the computer executable instructions cause the at least one processor to perform operations, and the operations comprise:
receiving physiological signals;
generating a plurality of parameters of heart rate variability based on the physiological signals; and
determining the fatigue index based on the plurality of parameters of heart rate variability.

12. The apparatus of claim 11, further comprising a sensor, wherein the physiological signals are received from the sensor.

13. The apparatus of claim 11, further comprising a communication module, wherein the physiological signals are received from the communication module.

14. The apparatus of claim 11, wherein the operations further comprise:
determining whether the physiological signals received in a first time period are obtained from a first phase or a second phase;
generating a low frequency (LF) power and a high frequency (HF) power for physiological signals received in the first time period; and
generating a ratio of the LF power to the HF power.

15. The apparatus of claim 14, wherein the operations further comprise:
generating a first disorder ratio of the ratio of the LF power to the HF power in a first phase;
generating a first average value of the ratio of the LF power to the HF power in a first phase; and
generating a second disorder ratio of the ratio of the LF power to the HF power in a second phase,
wherein the first phase and the second phase include multiple first time periods.

16. The apparatus of claim 15, wherein the operations further comprise:
determining a belonging group of the received physiological signals received in a second time period, wherein the second time period includes the first phase and the second phase; and
determining a fatigue index based on the belonging group of the received physiological signals.

17. The apparatus of claim 16, wherein the operations further comprise:
determining a first sub-group based on the first disorder ratio;
determining a second sub-group based on the first average value;
determining a third sub-group based on the second disorder ratio; and
determining, based on the first, second, and third sub-groups, the belonging group of the received physiological signals received in the second time period.

18. The apparatus of claim 17, wherein the operations further comprise:
receiving a feedback fatigue index;
adjusting a threshold of determining the belonging group of the received physiological signals received in the second time period; and
adjusting a shifting value of the belonging group.

19. The apparatus of claim 18, further comprising an input module, wherein the feedback fatigue index is received from the input module.

20. The apparatus of claim 18, further comprising a communication module, wherein the feedback fatigue index is received from the communication module.
